(19) United States
(12) Patent Application Publication
(10) Pub. No.: US 2007/0049638 A1
(43) Pub. Date: Mar. 1, 2007

(54) Polymorphic forms of 3-amino-1-propanesulfonic acid

(75) Inventor: France Guay, Boucherville (CA)

Correspondence Address: LAHIVE & COCKFIELD, LLP, ONE POST OFFICE SQUARE, BOSTON, MA, 02109-2127, US

(73) Assignee: Neurochem (International) Limited, Lausanne (CH)

(21) Appl. No.: 11/487,161

(22) Filed: Jul. 14, 2006

Related U.S. Application Data
(60) Provisional application No. 60/701,756, filed on Jul. 21, 2005.

(51) Int. Cl. A61K 31/185; C07C 309/13

(52) U.S. Cl. 514/553; 562/104

(57) ABSTRACT

Polymorphic forms of 3-amino-1-propanesulfonic acid are described.

### RELATED APPLICATION

This application claims priority to U.S. Provisional Patent Application Ser. No. 60/701,756, filed on Jul. 21, 2005; the entire contents of which are hereby incorporated herein by reference.

### BACKGROUND OF THE INVENTION

3-amino-1-propanesulfonic acid is disclosed in International Patent Publication No. WO 96/28187 to Kisilevsky et al. entitled “Methods for Treating Amyloidosis.”

### SUMMARY OF THE INVENTION

The invention pertains, at least in part, to the discovery that 3-amino-1-propanesulfonic acid may exist in at least two polymorphic forms, e.g., Form A and Form B.

In one embodiment, the invention pertains, at least in part, to crystalline 3-amino-1-propanesulfonic acid in polymorphic Form A.

In another embodiment, the invention pertains, at least in part, to crystalline 3-amino-1-propanesulfonic acid in polymorphic Form B.

In yet another embodiment, the invention also includes crystalline 3-amino-1-propanesulfonic acid in a mixture of Form A and Form B.

The invention also pertains, at least in part, to substantially pure crystalline 3-amino-1-propanesulfonic acid in polymorphic Form A.

The invention also pertains, at least in part, to substantially pure crystalline 3-amino-1-propanesulfonic acid in polymorphic Form B.

In yet another embodiment, the invention also pertains, at least in part, to pharmaceutical compositions comprising crystalline 3-amino-1-propanesulfonic acid in polymorphic Form A.

In another embodiment, the invention also pertains, at least in part, to pharmaceutical compositions comprising crystalline 3-amino-1-propanesulfonic acid in polymorphic Form B.

In another embodiment, the invention also pertains, at least in part, to pharmaceutical compositions comprising crystalline 3-amino-1-propanesulfonic acid in a mixture of polymorphic Form A and Form B.

In yet another embodiment, the invention pertains to a method for treating an Aβ-amyloid related disease in a subject, by administering to the subject, in need thereof, an effective amount of a crystalline 3-amino-1-propanesulfonic acid of polymorphic form A, such that the Aβ-amyloid related disease is treated in the subject.

In yet another embodiment, the invention pertains to a method for treating an Aβ-amyloid related disease in a subject, by administering to the subject, in need thereof, an effective amount of a crystalline 3-amino-1-propanesulfonic acid of polymorphic form B, such that the Aβ-amyloid related disease is treated in the subject.

In yet another embodiment, the invention pertains to a method for treating an Aβ-amyloid related disease in a subject, by administering to the subject, in need thereof, an effective amount of crystalline 3-amino-1-propanesulfonic acid in a mixture of polymorphic Form A and Form B, such that the Aβ-amyloid related disease is treated in the subject.

### BRIEF DESCRIPTION OF THE DRAWINGS

FIG. 1 is an X-ray powder diffraction (XRPD) pattern for 3-amino-1-propanesulfonic acid, Form A.

FIG. 2 is an FT-IR spectrum of 3-amino-1-propanesulfonic acid, Form A.

FIG. 3 is an FT-Raman spectrum of 3-amino-1-propanesulfonic acid, Form A.

FIG. 4 is an X-ray powder diffraction pattern for 3-amino-1-propanesulfonic acid, Form B.

FIG. 5 is an FT-IR spectrum of 3-amino-1-propanesulfonic acid, Form B.

FIG. 6 is an FT-Raman spectrum of 3-amino-1-propanesulfonic acid, Form B.

FIG. 7 is an X-ray powder diffraction pattern for a mixture of 3-amino-1-propanesulfonic acid, Form A and Form B.

### DETAILED DESCRIPTION OF THE INVENTION

The invention pertains, at least in part, to the discovery that 3-amino-1-propanesulfonic acid may exist in two polymorphic forms, Form A and Form B. 3-amino-1-propanesulfonic acid (homotaurine) is typically white powder at room temperature. In addition to 3-amino-1-propanesulfonic acid (the free acid), the invention also pertains to pharmaceutically acceptable salts and hydrated forms of the compound.

Variations in the polymorphic form of a compound may affect the physical and pharmaceutical properties of the compound. For example, solubility, melting point, density, hardness, crystal shape, optical and electrical properties, vapor pressure, stability, etc., may all vary with the polymorphic form (Remington's Pharmaceutical Sciences, 18th Edition, Mack Publishing Co. (1990), Chapter 75, pages 1439-1443). In some cases it could be advantageous to control polymorphic forms to provide consistent pharmaceutical compositions.

Before a compound can be commercialized, a process for its bulk manufacture must be developed that reliably provides a uniform and highly pure grade of the compound. Further, the process must deliver a form of the compound that can be suitably formulated for convenient dosage to patients and which is chemically and physically stable over long periods in that formulation. One crystalline form of a compound may have advantages over an amorphous form or another crystalline form in several respects. Further, one crystalline form is usually more stable than an amorphous form or other crystalline forms, both before and during formulation and during subsequent storage. There is no generally applicable method for preparing crystalline forms of a material. Indeed, it is impossible to know, from the outset, whether crystalline forms of a given compound exists. Where it turns out that a compound can be crystallized, extensive experimentation is usually required before a process is identified from which a particular crystalline form can be isolated. The correct combination of several independently variable conditions (for example, solvent concentration, solvent composition, temperature, cooling rate) must be identified empirically through trial and error with no guarantee of success. It is expected, however, that the polymorphic forms of the invention, e.g., Form A, Form B, and mixtures thereof, are useful for all the same uses previously described for 3-amino-1-propanesulfonic acid.

In one embodiment, the invention pertains to crystalline 3-amino-1-propanesulfonic acid in polymorphic Form A. The invention also pertains to crystalline 3-amino-1-propanesulfonic acid in polymorphic Form B.

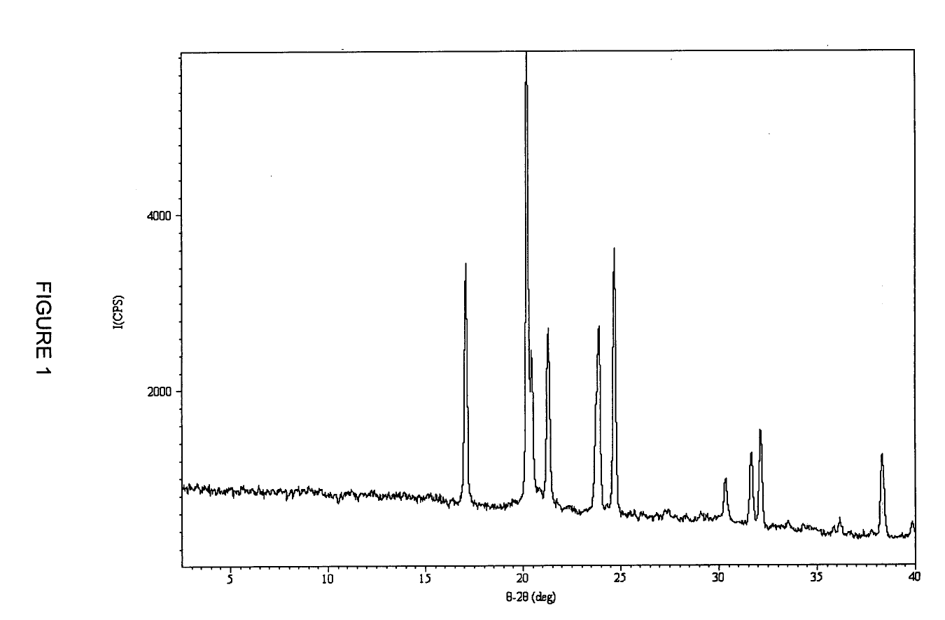

The term “crystalline” refers to 3-amino-1-propanesulfonic acid in the solid form, wherein a portion of the 3-amino-1-propanesulfonic acid molecules are in a crystal lattice. It also refers to a solid, substantially non-amorphous form of 3-amino-1-propanesulfonic acid which can be analyzed by X-ray powder diffraction (XRPD) to obtain a pattern similar to Form A, Form B, or Form A and B, as shown in FIGS. 1, 4, and 7, respectively.

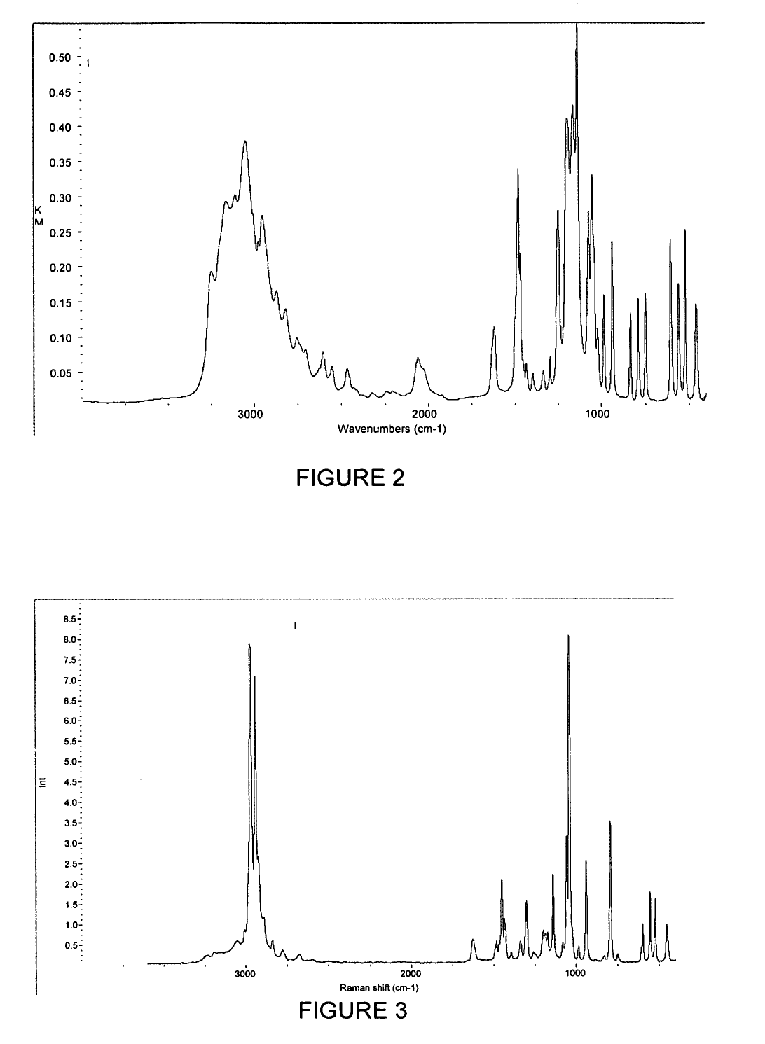

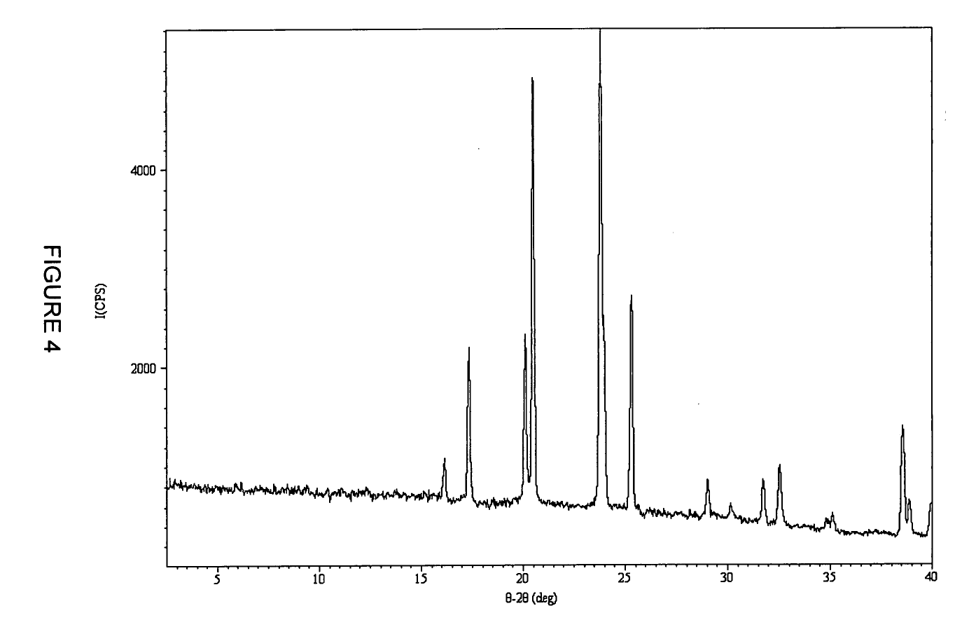

The term “polymorphic Form A” refers to a polymorphic form of 3-amino-1-propanesulfonic acid, which can be characterized by the XRPD pattern shown in FIG. 1. Form A is also further characterized by the FT-IR spectrum shown in FIG. 2 and the FT-Raman spectrum shown in FIG. 3. Form A can be synthesized using the methods described in Examples 1-3.

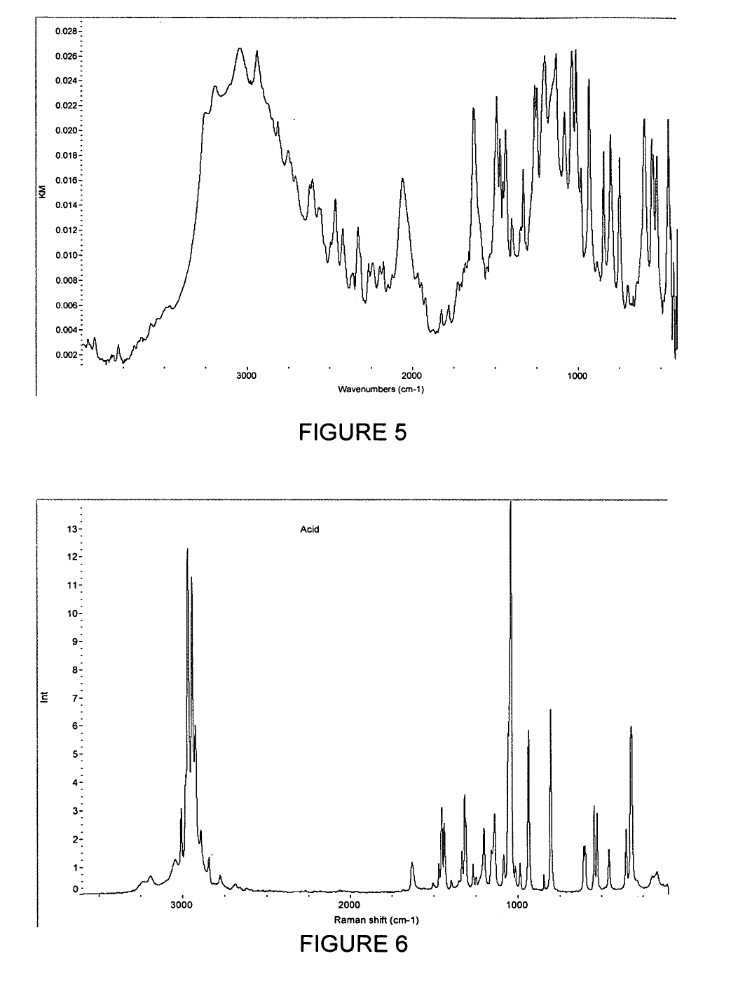

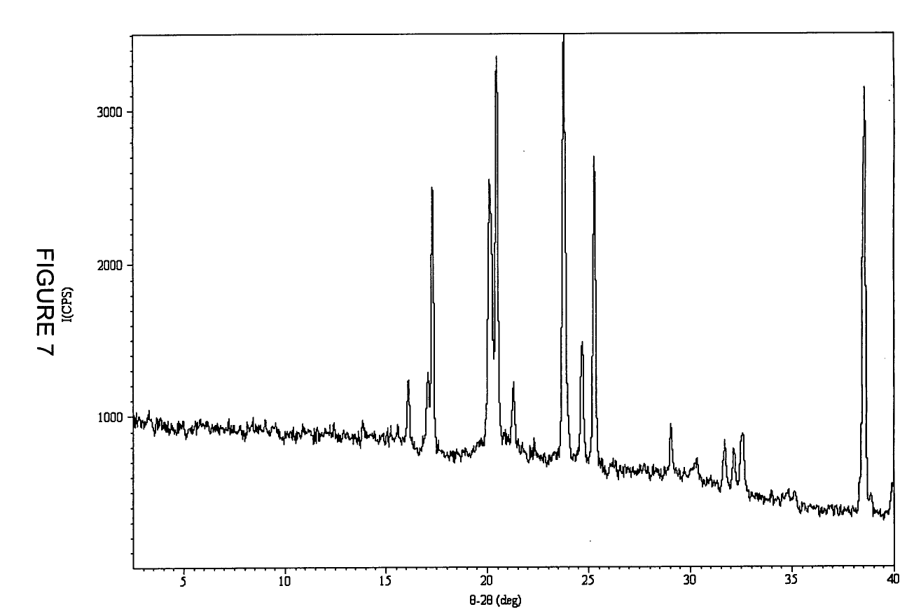

The term “polymorphic Form B” refers to a polymorphic form of 3-amino-1-propanesulfonic acid, which can be characterized by the XRPD pattern shown in FIG. 4. Form B is also further characterized by the FT-IR spectrum shown in FIG. 5 and the FT-Raman spectrum shown in FIG. 6. Form B can be synthesized using the methods described in Examples 4-6. It can also be generated from Form A using the method described in Example 7.

Form A and Form B can be distinguished from one another by peaks unique to Form A or Form B, using one of more of the techniques described above or in the Examples. For XRPD, exemplary unique peaks may be selected such that no other peak position is within ±0.2 °2θ. Examples of unique XRPD peaks are shown in Table 1. Accordingly, in one embodiment, the 3-amino-1-propanesulfonic acid is characterized by XRPD peaks shown in Table 1. The values in Table 1 are rounded to one decimal place.

TABLE 1

| Form A (°2θ) | Form B (°2θ) |
|---|---|
| 17.1 | 17.3 |
| 21.3 | 25.3 |
| 24.7 | — |

In a further embodiment, crystalline 3-amino-1-propanesulfonic acid in polymorphic Form A has XRPD peaks at one or more of the following °2θ values: 17.1, 21.3, and 24.7. In another further embodiment, crystalline 3-amino-1-propanesulfonic acid in polymorphic Form B has XRPD peaks at one or more of the following °2θ values: 17.3 and 25.3. Methodology for performing XRPD is described in further detail in Example 9.

For FT-IR, unique peaks were selected such that no other peak was within 4 cm⁻¹. Exemplary unique FT-IR peaks for each of Form A and Form B are shown in Table 2. Accordingly, in one embodiment, the 3-amino-1-propanesulfonic acid is characterized by FT-IR peaks at one or more of the wavelengths shown in Table 2.

TABLE 2

| Form A (cm⁻¹) | Form B (cm⁻¹) |
|---|---|
| 789 | 803 |
| 833 | 843 |

In another further embodiment, crystalline 3-amino-1-propanesulfonic acid in polymorphic Form A has FT-IR peaks at one or more of the following wavelengths: 789 cm⁻¹ and 833 cm⁻¹. In another further embodiment, crystalline 3-amino-1-propanesulfonic acid in polymorphic Form B has a FT-IR peaks at one or more of the following wavelengths: 803 cm⁻¹ and 843 cm⁻¹. The methodology for performing FT-IR spectroscopy is described in further detail in Example 10.

For FT-Raman, unique peaks were selected such that no other peak is within 4 cm⁻¹. Examples of unique FT-Raman peak for crystalline 3-amino-1-propanesulfonic acid in Form A include 790 cm⁻¹ and for Form B, 802 cm⁻¹. FT-Raman spectroscopy is described in greater detail in Example 11.

Form B is believed to be the more thermodynamically stable form between about 5 and about 60° C. Form A is believed to be the kinetically favored form and, in general, is generated from fast timescale experiments. Thus in general, without wishing to be bound by theory, slower processes will favor the production of Form B. For example, slow addition of solvent, slow cooling rate and/or mixing will tend to favor the production of Form B, whereas fast solvent addition, fast cooling and/or minimal mixing time will favor production of Form A.

In another embodiment, the invention pertains to crystalline 3-amino-1-propanesulfonic acid in a mixture of Form A and Form B. The mixture of polymorphic Form A and Form B can be in any proportion less than 90% (by weight) of pure Form A or pure Form B. In one embodiment, the mixture comprises about 11-15%, about 16-20%, about 21-25%, about 26-30%, about 31-35%, about 36-40%, about 41-45%, about 46-50%, about 51-55%, about 56-60%, about 61-65%, about 66-70%, about 71-75%, about 76-80%, about 81-85%, or about 86-89% of pure Form A. In another embodiment, the mixture comprises about 10-14%, about 15-19%, about 20-24%, about 25-29%, about 30-34%, about 35-39%, about 40-44%, about 45-49%, about 50-54%, about 55-59%, about 60-64%, about 65-69%, about 70-74%, about 75-79%, about 80-84%, or about 85-89% of pure Form B. Mixtures of Form A and Form B can be synthesized using the methods described in Example 8.

In another embodiment, the invention pertains to a mixture of polymorphic Form A and Form B that it is enriched for Form A. For example, a mixture enriched for Form A comprises about 60 to about 89% of Form A.

In another embodiment, the invention pertains to a mixture of polymorphic Form B and Form A that it is enriched for Form B. For example, a mixture enriched for Form B comprises about 60 to about 89% of Form B.

In another embodiment, the invention pertains to substantially pure crystalline 3-amino-1-propanesulfonic acid in polymorphic Form A or Form B.

The term “substantially pure” refers to compositions which can be determined to comprise at least 90% (by weight) of pure crystalline 3-amino-1-propanesulfonic acid in the desired polymorphic form (e.g., Form A or Form B). In a further embodiment, the composition comprises at least 90% or greater, 91% or greater, 92% or greater, 93% or greater, 94% or greater, 95% or greater, 96% or greater, 97% or greater, 98% or greater, or 99% or greater of the desired polymorphic form, e.g., Form A or Form B. The remaining impurities may be other polymorphic forms of 3-amino-1-propanesulfonic acid or other impurities, e.g., impurities resulting from the synthesis, production, packaging, formulation, etc. of the compound.

The term “about” refers to within 10%, preferably within 5%, and more preferably within 1% of a given value or range. The term “about” also includes within an acceptable standard error of the mean, when considered by one of ordinary skill in the art.

In another embodiment, the invention pertains to a method for treating an Aβ-amyloid related disease in a subject, by administering to the subject, in need thereof, an effective amount of a crystalline 3-amino-1-propanesulfonic acid, such that the Aβ-amyloid related disease is treated in the subject.

The term “amyloid” refers to amyloidogenic proteins, peptides, or fragments thereof which can be soluble (e.g., monomeric or oligomeric) or insoluble (e.g., having fibrillary structure or in amyloid plaque). See, e.g., M P Lambert, et al., Proc. Nat'l Acad. Sci. USA 95, 6448-53 (1998). “Amyloidosis” or “amyloid disease” or “amyloid-related disease” refers to a pathological condition characterized by the presence of amyloid fibers. “Amyloid” is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra.

The terms “Aβ-amyloid related diseases” or “amyloid-β diseases” refer to diseases or disorders which are associated with Aβ amyloidosis or are related to the undesirable formation and/or deposition of amyloid-β. Aβ-amyloid related diseases includes those diseases, disorders, conditions, pathologies, and other abnormalities of the structure or function of the brain, including components thereof, in which the causative agent is amyloid. Local deposition of amyloid is common in the brain, particularly in elderly individuals. The area of the brain affected in an amyloid-β disease may be the stroma including the vasculature or the parenchyma including functional or anatomical regions, or neurons themselves. The most frequent type of amyloid in the brain is composed primarily of Aβ peptide fibrils, resulting in dementia associated with e.g. Alzheimer's disease. A subject need not have received a definitive diagnosis of a specifically recognized amyloid-β disease.

Amyloid-β peptide (Aβ) is a 39-43 amino acid peptide derived by proteolysis from a large protein known as Beta Amyloid Precursor Protein (“βAPP”). Mutations in βAPP result in familial forms of Alzheimer's disease, Down's syndrome, cerebral amyloid angiopathy (e.g. hereditary cerebral hemorrhage) and senile dementia, characterized by cerebral deposition of plaques composed of Aβ fibrils and other components, which are described in further detail below. Known mutations in APP associated with Alzheimer's disease occur proximate to the cleavage sites of β or γ-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase in the amount of the 42/43 amino acid form of Aβ generated from APP. The familial form of Alzheimer's disease represents only 10% of the subject population. In fact, the incidence of sporadic Alzheimer's disease greatly exceeds forms shown to be hereditary. Nevertheless, fibril peptides forming plaques are very similar in both types.

The structure and sequence of Aβ peptides of various lengths are well known in the art. Such peptides can be made according to methods known in the art, or extracted from the brain according to known methods (e.g., Glenner and Wong, Biochem. Biophys. Res. Comm. 129, 885-90 (1984); Glenner and Wong, Biochem. Biophys. Res. Comm. 122, 1131-35 (1984)). In addition, various forms of the peptides are commercially available.

As used herein, the terms “β amyloid,” “amyloid-β” and the like refer to amyloid β proteins or peptides, amyloid β precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ1-39, Aβ1-40, Aβ1-41, Aβ1-42, and Aβ1-43. For convenience of nomenclature, “Aβ1-42” may be referred to herein as “Aβ(1-42)” or simply as “Aβ42” or “Aβ₄₂” (and likewise for any other amyloid peptides discussed herein). As used herein, the terms “β amyloid,” “amyloid-β,” and “Aβ” are synonymous. Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque). See, e.g., M P Lambert, et al., Proc. Nat'l Acad. Sci. USA 95, 6448-53 (1998).

According to certain aspects of the invention, amyloid-β is a peptide having 39-43 amino-acids, or amyloid-β is an amyloidogenic peptide produced from βAPP. The Aβ-amyloid related diseases that are the subject of the present invention include, without limitation, age-related cognitive decline, early Alzheimer's disease as seen in Mild Cognitive Impairment (“MCI”), vascular dementia, or Alzheimer's disease (“AD”), which may be sporadic (non-hereditary) Alzheimer's disease or familial (hereditary) Alzheimer's disease. The Aβ-amyloid related disease may also be cerebral amyloid angiopathy (“CAA”) or hereditary cerebral hemorrhage. The Aβ-amyloid related disease may be senile dementia, Down's syndrome, inclusion body myositis (“IBM”), or age-related macular degeneration (“ARMD”).

Mild cognitive impairment (“MCI”) is a condition characterized by a state of mild but measurable impairment in thinking skills, which is not necessarily associated with the presence of dementia. MCI frequently, but not necessarily, precedes Alzheimer's disease. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to “Mild-to-Moderate Cognitive Impairment,” as is well understood in this art.

Cerebral amyloid angiopathy (“CAA”) refers to the specific deposition of amyloid fibrils in the walls of leptomingeal and cortical arteries, arterioles and in capillaries and veins. It is commonly associated with Alzheimer's disease, Down's syndrome and normal aging, as well as with a variety of familial conditions related to stroke or dementia (see Frangione, et al., Amyloid: J Protein Folding Disord. 8, Suppl. 1, 36-42 (2001)). CAA can occur sporadically or be hereditary. Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia. Cerebral amyloid angiopathy is known to be associated with cerebral hemorrhage (or hemorrhagic stroke).

Additionally, abnormal accumulation of APP and of amyloid-β protein in muscle fibers has been implicated in the pathology of sporadic inclusion body myositis (“IBM”) (Askanas, et al., Proc. Natl. Acad. Sci. USA 93, 1314-19 (1996); Askanas, et al., Current Opinion in Rheumatology 7, 486-96 (1995)). Accordingly, the compounds and compositions of the invention can be used prophylactically or therapeutically in the treatment of disorders in which amyloid-β protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson, et al., Proc. Natl. Acad. Sci. USA 99(18), 11830-5 (2002)). Therefore, the invention also relates to the treatment of age-related macular degeneration.

APP is expressed and constitutively catabolized in most cells. The dominant catabolic pathway appears to be cleavage of APP within the Aβ sequence by the α-secretase enzyme, leading to release of a soluble ectodomain fragment known as APPsα In contrast to this non-amyloidogenic pathway, APP can also be cleaved by enzymes known as β- and γ-secretase at the N- and C-termini of the Aβ, respectively, followed by release of Aβ into the extracellular space. To date, BACE has been identified as β-secretase (Vasser, et al., Science 286:735-741, 1999) and presenilins have been implicated in γ-secretase activity (De Strooper, et al., Nature 391, 387-90 (1998)).

The 39-43 amino acid Aβ peptide is produced by sequential proteolytic cleavage of the amyloid precursor protein (APP) by the enzyme(s) β and γ secretases. Although Aβ40 is the predominant form produced, 5-7% of total Aβ exists as Aβ42 (Cappai et al., Int. J. Biochem. Cell Biol. 31. 885-89 (1999)). The length of the Aβ peptide appears to dramatically alter its biochemical/biophysical properties. Specifically, the additional two amino acids at the C-terminus of Aβ42 are very hydrophobic, presumably increasing the propensity of Aβ42 to aggregate. For example, Jarrett, et al. demonstrated that Aβ42 aggregates very rapidly in vitro compared to Aβ40, suggesting that the longer forms of Aβ may be important pathological proteins that are involved in the initial seeding of the neuritic plaques in Alzheimer's disease (Jarrett, et al., Biochemistry 32, 4693-97 (1993); Jarrett, et al., Ann. N.Y. Acad. Sci. 695, 144-48 (1993)).

This hypothesis has been further substantiated by the recent analysis of the contributions of specific forms of Aβ in cases of genetic familial forms of Alzheimer's disease (“FAD”). For example, the “London” mutant form of APP (APPV7171) linked to FAD selectively increases the production of Aβ 42/43 forms versus Aβ 40 (Suzuki, et al., Science 264, 1336-40 (1994)) while the “Swedish” mutant form of APP (APPK670N/M671L) increases levels of both Aβ40 and Aβ42/43 (Citron, et al., Nature 360, 672-674 (1992); Cai, et al., Science 259, 514-16, (1993)). Also, it has been observed that FAD-linked mutations in the Presenilin-1 (“PS 1”) or Presenilin-2 (“PS2”) genes will lead to a selective increase in Aβ42/43 production but not Aβ40 (Borchelt, et al., Neuron 17, 1005-13 (1996)). This finding was corroborated in transgenic mouse models expressing PS mutants that demonstrate a selective increase in brain Aβ42 (Borchelt, op cit.; Duff, et al., Neurodegeneration 5(4), 293-98 (1996)). Thus the leading hypothesis regarding the etiology of Alzheimer's disease is that an increase in Aβ42 brain concentration due to an increased production and release of Aβ42 or a decrease in clearance (degradation or brain clearance) is a causative event in the disease pathology.

Multiple mutation sites in either Aβ or the APP gene have been identified and are clinically associated with either dementia or cerebral hemorrhage. In addition to the FAD mutations mentioned above, exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia. CAA may also be sporadic.

The term “treating” includes the application or administration of a composition or compound of the invention to a subject, or application or administration of a composition or compound of the invention to a cell or tissue from a subject, who has an Aβ-amyloid related disease or condition, has a symptom of such a disease or condition, or is at risk of (or susceptible to) such a disease or condition, with the purpose of curing, healing, alleviating, relieving, altering, remedying, ameliorating, preventing, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of dementia. Treatment may be therapeutic or prophylactic. The treatment or amelioration of symptoms can be based on objective or subjective parameters; including the results of a physical examination, a psychiatric evaluation, or a cognition test such as CDR, MMSE, ADAS-Cog, or another test known in the art. For example, the methods of the invention successfully treat a subject's dementia by slowing the rate of or lessening the extent of cognitive decline.

The term “subject” includes living organisms in which Aβ-amyloidosis can occur, or which are susceptible to Aβ-amyloid diseases, e.g., Alzheimer's disease, etc. Examples of subjects include humans, chickens, ducks, peking ducks, geese, monkeys, deer, cows, rabbits, sheep, goats, dogs, cats, mice, rats, and transgenic species thereof. Administration of the compositions or compounds of the present invention to a subject to be treated can be carried out using known procedures, at dosages and for periods of time effective to treat or prevent an Aβ-amyloid related disease, e.g. Alzheimer's disease, or to e.g. modulate amyloid aggregation or amyloid-induced toxicity or to stabilize cognitive decline in the subject as further described herein.

In certain embodiments of the invention, the subject is in need of treatment by the methods of the invention, and is selected for treatment based on this need. A subject in need of treatment is art-recognized, and includes subjects that have been identified as having a disease or disorder related to Aβ-amyloid-deposition or amyloidosis, has a symptom of such a disease or disorder, or is at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

In still a further embodiment, the subject is shown to be at risk by a cognitive test such as Clinical Dementia Rating (“CDR”), Alzheimer's Disease Assessment Scale-Cognition (“ADAS-Cog”), or Mini-Mental State Examination (“MMSE”). The subject may exhibit a below average score on a cognitive test, as compared to a historical control of similar age and educational background. The subject may also exhibit a reduction in score as compared to previous scores of the subject on the same or similar cognition tests.

In determining the CDR, a subject is typically assessed and rated in each of six cognitive and behavioural categories: memory, orientation, judgement and problem solving, community affairs, home and hobbies, and personal care. The assessment may include historical information provided by the subject, or preferably, a corroborator who knows the subject well. The subject is assessed and rated in each of these areas and the overall rating, (0, 0.5, 1.0, 2.0 or 3.0) determined. A rating of 0 is considered normal. A rating of 1.0 is considered to correspond to mild dementia. A subject with a CDR of 0.5 is characterized by mild consistent forgetfulness, partial recollection of events and “benign” forgetfulness. In one embodiment the subject is assessed with a rating on the CDR of above 0, of above about 0.5, of above about 1.0, of above about 1.5, of above about 2.0, of above about 2.5, or at about 3.0.

Another test is the Mini-Mental State Examination (MMSE), as described by Folstein “Mini-mental state. A practical method for grading the cognitive state of patients for the clinician.” J. Psychiatr. Res. 12:189-198, 1975. The MMSE evaluates the presence of global intellectual deterioration. See also Folstein “Differential diagnosis of dementia. The clinical process.” Psychiatr Clin North Am. 20:45-57, 1997. The MMSE is a means to evaluate the onset of dementia and the presence of global intellectual deterioration, as seen in Alzheimer's disease and multi-infart dementia. The MMSE is scored from 1 to 30. The MMSE does not evaluate basic cognitive potential, as, for example, the so-called IQ test. Instead, it tests intellectual skills. A person of “normal” intellectual capabilities will score a “30” on the MMSE objective test (however, a person with a MMSE score of 30 could also score well below “normal” on an IQ test). See, e.g., Kaufer, J. Neuropsychiatry Clin. Neurosci. 10:55-63, 1998; Becke, Alzheimer Dis Assoc Disord. 12:54-57, 1998; Ellis, Arch. Neurol. 55:360-365, 1998; Magni, Int. Psychogeriatr. 8:127-134, 1996; Monsch, Acta Neurol. Scand. 92:145-150, 1995. In one embodiment, the subject scores below 30 at least once on the MMSE. In another embodiment, the subject scores below about 28, below about 26, below about 24, below about 22, below about 20, below about 18, below about 16, below about 14, below about 12, below about 10, below about 8, below about 6, below about 4, below about 2, or below about 1.

Another means to evaluate cognition, particularly Alzheimer's disease, is the Alzheimer's Disease Assessment Scale (ADAS-Cog), or a variation termed the Standardized Alzheimer's Disease Assessment Scale (SADAS). It is commonly used as an efficacy measure in clinical drug trials of Alzheimer's disease and related disorders characterized by cognitive decline. SADAS and ADAS-Cog were not designed to diagnose Alzheimer's disease; they are useful in characterizing symptoms of dementia and are a relatively sensitive indicator of dementia progression. (See, e.g., Doraiswamy, Neurology 48:1511-1517, 1997; and Standish, J. Am. Geriatr. Soc. 44:712-716, 1996.) Annual deterioration in untreated Alzheimer's disease patients is approximately 8 points per year (See, eg., Raskind, M Prim. Care Companion J Clin Psychiatry 2000 August; 2(4): 134-138).

The ADAS-cog is designed to measure, with the use of questionnaires, the progression and the severity of cognitive decline as seen in AD on a 70-point scale. The ADAS-cog scale quantifies the number of wrong answers. Consequently, a high score on the scale indicates a more severe case of cognitive decline. In one embodiment, a subject exhibits a score of greater than 0, greater than about 5, greater than about 10, greater than about 15, greater than about 20, greater than about 25, greater than about 30, greater than about 35, greater than about 40, greater than about 45, greater than about 50, greater than about 55, greater than about 60, greater than about 65, greater than about 68, or about 70.

In another embodiment, the subject exhibits no symptoms of Alzheimer's Disease. In another embodiment, the subject is a human who is at least 40 years of age and exhibits no symptoms of Alzheimer's Disease. In another embodiment, the subject is a human who is at least 40 years of age and exhibits one or more symptoms of Alzheimer's Disease.

In another embodiment, the subject has Mild Cognitive Impairment. In a further embodiment, the subject has a CDR rating of about 0.5. In another embodiment, the subject has early Alzheimer's disease. In another embodiment, the subject has cerebral amyloid angiopathy.

In another embodiment, the polymorphic compounds or compositions of the invention are administered at a therapeutically effective dosage sufficient to reduce the levels of amyloid β peptides in a subject's plasma or cerebrospinal fluid (CSF) from levels prior to treatment from about 10 to about 100 percent, or even about 50 to about 100 percent.

The amount of amyloid β peptide in the brain, CSF, blood, or plasma of a subject can be evaluated by enzyme-linked immunosorbent assay (“ELISA”) or quantitative immunoblotting test methods or by quantitative SELDI-TOF which are well known to those skilled in the art, such as is disclosed by Zhang, et al., J. Biol. Chem. 274, 8966-72 (1999) and Zhang, et al., Biochemistry 40, 5049-55 (2001). See also, A. K. Vehmas, et al., DNA Cell Biol. 20(11), 713-21 (2001), P. Lewczuk, et al., Rapid Commun. Mass Spectrom. 17(12), 1291-96 (2003); B. M. Austen, et al., J. Peptide Sci. 6, 459-69 (2000); and H. Davies, et al., BioTechniques 27, 1258-62 (1999). These tests are performed on samples of the brain or blood which have been prepared in a manner well known to one skilled in the art. Another example of a useful method for measuring levels of amyloid β peptides is by Europium immunoassay (EIA). See, e.g., WO 99/38498 at p. 11.

In another embodiment, the subject may have (or may be predisposed to developing or may be suspected of having or may be at risk of) e.g. Alzheimer's disease, dementia, vascular dementia, or senile dementia, Mild Cognitive Impairment, or early Alzheimer's disease. In addition to Alzheimer's disease, the subject may have e.g. another Aβ-amyloid related disease such as cerebral amyloid angiopathy, or the subject may have amyloid deposits, especially amyloid-β amyloid deposits in the brain. In still a further embodiment, the subject is shown to be at risk by a diagnostic brain imaging technique, for example, one that measures brain activity, plaque deposition, or brain atrophy.

In another embodiment, the invention pertains to a method for improving cognition in a subject suffering from an Aβ-amyloid related disease. The method includes administering an effective amount of a polymorphic compound or composition of the invention, such that the subject's cognition is stabilized or improved. The subject's cognition can be tested using methods known in the art such as the Clinical Dementia Rating (“CDR”), Mini-Mental State Examination (“MMSE”), and the Alzheimer's Disease Assessment Scale-Cognition (“ADAS-Cog”).

In one embodiment, the polymorphic compounds or compositions of the invention are administered at a therapeutically effective dosage sufficient to maintain a subject's CDR rating at its base line rating or at 0. In another embodiment, the polymorphic compounds or compositions of the invention are administered at a therapeutically effective dosage sufficient to decrease (i.e. improve) a subject's CDR rating by about 0.25 or more, about 0.5 or more, about 1.0 or more, about 1.5 or more, about 2.0 or more, about 2.5 or more, or about 3.0 or more. In another embodiment, the polymorphic compounds or compositions of the invention are administered at a therapeutically effective dosage sufficient to reduce the rate of the increase of a subject's CDR rating as compared to historical controls. In another embodiment, the polymorphic compounds or compositions of the invention are administered at a therapeutically effective dosage sufficient to reduce the rate of increase of a subject's CDR rating by about 5% or more, about 10% or more, about 20% or more, about 25% or more, about 30% or more, about 40% or more, about 50% or more, about 60% or more, about 70% or more, about 80% or more, about 90% or more, or about 100%, of the increase of the historical or untreated controls.

In another embodiment, the polymorphic compounds or compositions of the invention are administered at a therapeutically effective dosage sufficient to maintain a subject's score on the MMSE. The polymorphic compounds or compositions of the invention may be administered at a therapeutically effective dosage sufficient to increase a subject's MMSE score by about 1, about 2, about 3, about 4, about 5, about 7.5, about 10, about 12.5, about 15, about 17.5, about 20, or about 25 points. In another embodiment, the polymorphic compounds or compositions of the invention are administered at a therapeutically effective dosage sufficient to reduce the rate of the decrease of a subject's MMSE score as compared to historical controls. In another embodiment, the polymorphic compounds or compositions of the invention are administered at a therapeutically effective dosage sufficient to reduce the rate of decrease of a subject's MMSE score by about 5% or more, about 10% or more, about 20% or more, about 25% or more, about 30% or more, about 40% or more, about 50% or more, about 60% or more, about 70% or more, about 80% or more, about 90% or more or about 100% or more, of the decrease of the historical or untreated controls.

In yet another embodiment, the polymorphic compounds or compositions of the invention are administered at a therapeutically effective dosage sufficient to maintain a subject's score on the ADAS-Cog. In another embodiment, the polymorphic compounds or compositions of the invention are administered at a therapeutically effective dosage sufficient to decrease a subject's ADAS-Cog score by about 1 point or greater, by about 2 points or greater, by about 3 points or greater, by about 4 points or greater, by about 5 points or greater, by about 7.5 points or greater, by about 10 points or greater, by about 12.5 points or greater, by about 15 points or greater, by about 17.5 points or greater, by about 20 points or greater, or by about 25 points or greater. The polymorphic compounds or compositions of the invention may also be administered at a therapeutically effective dosage sufficient to reduce the rate of the increase of a subject's ADAS-Cog score as compared to historical controls. In another embodiment, the polymorphic compounds or compositions of the invention are administered at a therapeutically effective dosage sufficient to reduce the rate of increase of a subject's ADAS-Cog score by about 5% or more, about 10% or more, about 20% or more, about 25% or more, about 30% or more, about 40% or more, about 50% or more, about 60% or more, about 70% or more, about 80% or more, about 90% or more or about 100% of the increase of the historical or untreated controls. In a further embodiment, the polymorphic compounds or compositions of the invention may be administered at a therapeutically effective dosage sufficient to treat, slow or stop an Aβ-amyloid related disease associated with cognition such that the subject's cognition as measured by ADAS-Cog remains constant over a year. “Constant” includes fluctuations of no more than 2 points. Remaining constant includes fluctuations of two points or less in either direction.

In a further embodiment, the invention pertains to a pharmaceutical composition comprising crystalline 3-amino-1-propanesulfonic acid, as described above, in polymorphic Form A, Form B, or a mixture of Form A and Form B. The pharmaceutical composition may further comprise a pharmaceutically acceptable carrier. In a further embodiment, the crystalline 3-amino-1-propanesulfonic acid polymorph of the invention may be provided in an effective amount to treat Aβ-amyloid related disease, such as, for example, Alzheimer's disease, CAA, etc.

Pharmaceutical compositions comprising the 3-amino-1-propanesulfonic acid polymorphs of the invention can be orally administered, for example, with an inert diluent or an assimilable edible carrier. The polymorphic compound of the invention and other ingredients may also be enclosed in a hard or soft shell gelatin capsule, compressed into tablets, or incorporated directly into the subject's diet. For oral therapeutic administration, the compound may be incorporated with excipients and used in the form of ingestible tablets, buccal tablets, troches, capsules, wafers, and the like. The percentage of the compound in the compositions and preparations may, of course, be varied. The amount of the compound of the invention in such therapeutically effective compositions is such that a suitable dosage will be obtained. Exemplary formulations of the polymorphic compounds of the invention for oral administration are shown in Examples 12-17.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of the polymorphic compound calculated to produce the desired therapeutic effect in association with the required pharmaceutical vehicle. The specification for the dosage unit forms of the invention are dictated by and directly dependent on (a) the unique characteristics of the polymorphic compound and the particular therapeutic effect to be achieved, and (b) the limitations inherent in the art of compounding such a polymorphic compound for the treatment of amyloid deposition in subjects.

The present invention therefore includes pharmaceutical formulations comprising the polymorphic compound of the invention, in pharmaceutically acceptable vehicles for oral and parenteral administration. In accordance with the present invention, a polymorphic compound of the invention may be administered orally or through inhalation as a solid.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents as are known in the art may also be included.

In one embodiment, the polymorphic compounds or compositions of the invention are administered at a therapeutically effective dosage sufficient to inhibit Aβ-amyloid deposition in a subject and/or treat a Aβ-amyloid related disease in a subject. An “effective” dosage may inhibit Aβ-amyloid deposition by, for example, at least about 20%, or by at least about 40%, or even by at least about 60%, or by at least about 80% relative to untreated subjects. In another embodiment, a “therapeutically effective” dosage stabilizes cognitive function or prevents a further decrease in cognitive function (i.e., preventing, slowing, or stopping disease progression) in a subject, e.g., a subject having Alzheimer's disease, CAA, etc.

Furthermore, the polymorphic compounds or compositions may be administered at a therapeutically effective dosage sufficient to decrease deposition in a subject of amyloid protein, e.g., Aβ40 or Aβ42. A therapeutically effective dosage decreases amyloid deposition by, for example, at least about 15%, or by at least about 40%, or even by at least 60%, or at least by about 80% relative to untreated subjects.

It is understood that appropriate doses depend upon a number of factors within the ken of the ordinarily skilled physician, veterinarian, or researcher. The dose(s) of the polymorphic compound will vary, for example, depending upon the identity, size, and condition of the subject or sample being treated, further depending upon the route by which the composition is to be administered, if applicable, and the effect which the practitioner desires the polymorphic compound to have upon the subject. Exemplary doses include milligram or microgram amounts of the polymorphic compound per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligram per kilogram, about 1 milligram per kilogram to about 10 milligrams per kilogram, or about 3 milligrams per kilogram to about 5 milligrams per kilogram). It is furthermore understood that appropriate doses depend upon the potency. Such appropriate doses may be determined using the assays described herein. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, and any drug combination.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The contents of all references, issued patents, and published patent applications cited throughout this application are hereby incorporated by reference. The invention is further illustrated by the following examples, which should not be construed as further limiting.

### EXEMPLIFICATION OF THE INVENTION

### Example 1

### Preparation of 3-Amino-1-Propanesulfonic Acid, Form A

3-Amino-1-propanesulfonic acid (˜30 mg) was added to water (0.1 mL) and 2,2,2-trifluoroethanol (0.2 mL). The mixture was warmed to ˜48° C. with agitation. The resulting solution was filtered through 0.2 μm nylon filter into a clean vial, which was warmed on a hotplate at 60° C. The hotplate was subsequently switched off. A small amount of precipitation was noted when the sample had cooled to ambient temperature and the sample was then refrigerated. Solids were collected by vacuum filtration to afford form A.

### Example 2

### Preparation of 3-Amino-1-Propanesulfonic Acid, Form A

3-Amino-1-propanesulfonic acid (˜30 mg) was dissolved in water (0.2 mL) with sonication. 1,4-Dioxane was added (0.4 mL) causing immediate precipitation. The solids were collected by vacuum filtration to afford form A.

### Example 3

### Preparation of 3-Amino-1-Propanesulfonic Acid, Form A

3-Amino-1-propanesulfonic acid (0.1182 g) was dissolved in water (0.4 mL) with sonication. The solution was filtered through 0.21 μm nylon filter into a clean vial and isopropyl alcohol was added (0.6 mL) causing immediate precipitation. The solids were collected by vacuum filtration to afford form A.

### Example 4

### Preparation of 3-Amino-1-Propanesulfonic Acid, Form B

3-Amino-1-propanesulfonic acid (˜30 mg) was dissolved in water (0.15 mL) with sonication. The solution was then filtered through 0.2 μm nylon filter into a clean vial, which was then placed inside a larger vial containing acetone. The larger vial was capped and left under ambient conditions. Precipitates formed and the remaining solution was decanted and the solids allowed to dry in air to afford form B.

### Example 5

### Preparation of 3-Amino-1-Propanesulfonic Acid, Form B

3-Amino-1-propanesulfonic acid (˜31 mg) was dissolved in water (0.3 mL) with sonication and acetonitrile (0.2 mL) added. The solution was filtered through 0.2 μm nylon filter into a clean vial, which was then covered with Parafilm™ and perforated with holes. The solution was allowed to evaporate to dryness under ambient conditions, affording form B.

### Example 6

### Preparation of 3-Amino-1-Propanesulfonic Acid, Form B

3-Amino-1-propanesulfonic acid (˜30 mg) was dissolved in water (0.15 mL) with sonication and methanol (0.25 mL) added. Some precipitation occurred and additional water (0.1 mL) was added. The solution was then filtered through 0.2 μm nylon filter into a clean vial, which was then covered with Parafilm™ and perforated with holes. The solution was allowed to evaporate to dryness under ambient conditions, affording form B.

### Example 7

### Generation of 3-Amino-1-Propanesulfonic Acid Form B from a Mixture of Forms A and B

A mixture of 3-amino-1-propanesulfonic acid form A (0.7654 g) and form B (0.7880 g) were added to ethanol (5 mL) and water (1.25 mL) in a flask. The slurry was placed in a water bath at 5° C. and stirred for four hours. Solids were collected by vacuum filtration to afford form B.

### Example 8

### Generation of 3-Amino-1-Propanesulfonic Acid Form A and B Mixture

3-Amino-1-propanesulfonic acid (˜31 mg) was added to water (0.2 mL) and 1,4-dioxane (0.2 mL) and the mixture warmed to ˜48° C. with agitation. The resulting solution was filtered through 0.2 μm nylon filter into a clean vial, which was warmed on a hotplate at 60° C. The hotplate was switched off and the sample allowed to cool to ambient temperature, and then refrigerated. Solids were collected by vacuum filtration to afford a mixture of forms A and B.

### Example 9

### X-Ray Powder Diffraction (XRPD) Studies of 3-Amino-1-Propanesulfonic Acid in Polymorphic Form A, Form B, and Form A and B

XRPD analyses were performed using an Inel XRG-3000™ diffractometer equipped with a CPS (Curved Position Sensitive) detector with a 2θ range of 120°. Real time data were collected using Cu—Kα radiation starting at approximately 4 °2θ at a resolution of 0.03 °2θ. The tube voltage and amperage were set to 40 kV and 30 mA, respectively. The monochromator slit was set at 5 mm by 80 μm or 160 μm. The pattern is displayed from 2.5-40 °2θ. Samples were prepared for analysis by packing them into thin-walled glass capillaries. Each capillary was mounted onto a goniometer head that is motorized to permit spinning of the capillary during data acquisition. The samples were analyzed for 5 minutes. Instrument calibration was performed using a silicon reference standard.

The XRPD diffraction patterns of 3-amino-1-propanesulfonic acid are shown in FIG. 1 (Form A), FIG. 4 (Form B) and FIG. 7 (Form A+B). The peaks from the XRPD for Form A and Form B are compared in Table 3. I/Io refers to the relative intensity of the peaks.

TABLE 3

| Form A | | | Form B | | |
|---|---|---|---|---|---|
| Peak No. | °2-Theta | I/Io | Peak No. | °2-Theta | I/Io |
| 1 | 17.1 | 52 | 1 | 16.1 | 7 |
| 2 | 20.2 | 100 | 2 | 17.3 | 31 |
| 3 | 20.4 | 33 | 3 | 20.1 | 34 |
| 4 | 21.3 | 39 | 4 | 20.5 | 87 |
| 5 | 23.8 | 42 | 5 | 23.8 | 100 |
| 6 | 24.7 | 61 | 6 | 25.3 | 47 |
| 7 | 30.3 | 10 | 7 | 29.0 | 8 |
| 8 | 31.6 | 18 | 8 | 30.1 | 3 |
| 9 | 32.1 | 24 | 9 | 31.7 | 10 |
| 10 | 36.1 | 4 | 10 | 32.5 | 13 |
| 11 | 38.3 | 22 | 11 | 35.1 | 4 |
| 12 | 39.8 | 4 | 12 | 38.5 | 26 |
| — | — | — | 13 | 38.8 | 9 |
| — | — | — | 14 | 39.9 | 8 |

### Example 10

### Fourier Transform Infrared Spectroscopy (FT-IR) Studies of 3-Amino-1-Propanesulfonic Acid in Polymorphic Form A and Form B

Infrared spectra were acquired on a Magna-IR 860® Fourier transform infrared (FT-IR) spectrophotometer (Thermo Nicolet™) equipped with an Ever-Glo™ mid/far IR source, an extended range potassium bromide (KBr) beamsplitter, and a deuterated triglycine sulfate (DTGS) detector. A diffuse reflectance accessory (the Collector™, Thermo Spectra-Tech) was used for sampling. Each spectrum represents 256 co-added scans collected at a spectral resolution of 4 cm⁻¹. Sample preparation consisted of physically mixing the sample with KBr and placing the sample into a 13-mm diameter cup and leveling material with a frosted glass slide. A background data set was acquired on a sample of KBr. A Log 1/R (R=reflectance) spectrum was acquired by taking a ratio of these two data sets against each other and was then converted to Kubelka-Munk units. Wavelength calibration was performed using polystyrene.

The FT-IR spectra of 3-amino-1-propanesulfonic acid are shown in FIG. 2 (Form A) and FIG. 5 (Form B). The peaks from the FT-IR spectra for Form A and Form B are listed in Table 4.

TABLE 4

| | |
|---|---|
| Form A (cm⁻¹) | Form B (cm⁻¹) |

| | | | |
|---|---|---|---|
| 746 | 2059 | 700 | 1629 |
| 789 | 2466 | 748 | 1778 |
| 833 | 2552 | 803 | 1825 |
| 935 | 2605 | 843 | 2058 |
| 985 | 2756 | 933 | 2174 |
| 1023 | 2954 | 985 | 2240 |
| 1051 | 3048 | 1013 | 2266 |
| 1072 | — | 1036 | 2329 |
| 1137 | — | 1081 | 2359 |
| 1163 | — | 1134 | 2422 |
| 1199 | — | 1201 | 2466 |
| 1247 | — | 1248 | 2606 |
| 1297 | — | 1261 | 2753 |
| 1339 | — | 1330 | 2816 |
| 1397 | — | 1400 | 2942 |
| 1435 | — | 1435 | 3045 |
| 1468 | — | 1451 | 3778 |
| 1480 | — | 1468 | 3922 |
| 1620 | — | 1491 | — |

### Example 11

### Fourier Transform Raman Spectroscopy (FT-Raman) Studies of 3-Amino-1-Propanesulfonic Acid in Polymorphic Form A and Form B

FT-Raman spectra were acquired on a Raman accessory module interfaced to a Magna 860® Fourier transform infrared (FT-IR) spectrophotometer (Thermo Nicolet™). This module uses an excitation wavelength of 1064 nm and an indium gallium arsenide (InGaAs) detector. Approximately 1 W or 0.711 W of Nd:YVO₄ laser power was used to irradiate the sample. The samples were prepared for analysis by placing the material in a glass tube and positioning the tube in the accessory. A total of 256 sample scans were collected from 3600-98 cm⁻¹ at a spectral resolution of 4 cm⁻¹, using Happ-Genzel apodization. Wavelength calibration was performed using sulfur and cyclohexane.

The FT-Raman spectra of 3-amino-1-propanesulfonic acid are shown in FIG. 3 (Form A) and FIG. 6 (Form B). The peaks from the FT-Raman spectra for Form A and Form B are listed in Table 5.

TABLE 5

| | |
|---|---|
| Form A (cm⁻¹) | Form B (cm⁻¹) |

| | | | |
|---|---|---|---|
| 790 | 2778 | 802 | 1435 |
| 936 | 2838 | 844 | 1451 |
| 985 | 2940 | 933 | 1468 |
| 1039 | 2969 | 985 | 1503 |
| 1057 | — | 1013 | 1630 |
| 1137 | — | 1036 | 2778 |
| 1173 | — | 1082 | 2844 |
| 1199 | — | 1136 | 2891 |
| 1300 | — | 1199 | 2923 |
| 1340 | — | 1247 | 2943 |
| 1395 | — | 1265 | 2967 |
| 1434 | — | 1308 | 3008 |
| 1451 | — | 1314 | 3044 |
| 1482 | — | 1331 | 3193 |
| 1626 | — | 1393 | — |

### Example 12

### Unit Formula of 100 mg Modified-Release Coated Tablets Containing 3-Amino-1-Propanesulfonic Acid, Form A

An example of a formulation of a 100 mg capsule of 3-amino-1-propanesulfonic acid, form A is described below.

Capsules of 100 mgs of 3-amino-1-propanesulfonic acid, form A, are manufactured using the formulation shown in Table 6. The coating is applied through several process steps using evaporation of purified water.

TABLE 6

| | | | Quantity per | Proportion |
|---|---|---|---|---|
| Ingredient | Grade | Function | tablet (mg) | (%) |
| Core: | | | | |
| 3-amino-1-propanesulfonic acid, Form A | MS* | Active ingredient | 100.00 | 28.6 |
| Silicated mycrocrystalline cellulose | NF | Glidant/Diluent | 140.35 | 40.1 |
| Dibasic calcium phosphate | USP | Filler | 63.80 | 18.2 |
| Hydroxypropylmethylcellulose (HPMC) | USP | Drug Release Modifier | 35.00 | 10.0 |
| Starch ® 1500 | NF | Binder/Desintegrant | 5.55 | 1.6 |
| Stearic acid powder | NF | Lubricant | 3.50 | 1.0 |
| Magnesium stearate | NF | Lubricant | 1.80 | 0.5 |
| Weight: | | | 350.00 | 100.0 |
| Coating: | | — | | |
| Opadry ® II White | MS* | Subcoat | 7.00 | 2.0 |
| Acryleze ® | MS* | Enteric Coat | 35.00 | 10.0 |
| Opadry ® II White | MS* | Topcoat | 7.00 | 2.0 |
| Total Weight: | | | 399.00 | 114.0 |

*MS: Manufacturer's Standard, NF: National Formulary; USP: United States Pharmacopoeia.

### Example 13

### Unit Formula of 100 mg Modified-Release Coated Tablets Containing 3-Amino-1-Propanesulfonic Acid, Form B

A pharmaceutical composition is formulated as described in Example 12 with 3-amino-1-propanesulfonic acid, form B, as the active ingredient.

### Example 14

### Unit Formula of 150 mg Modified-Release Coated Tablets Containing 3-Amino-1-Propanesulfonic Acid, Form A

An example of a formulation of a 150 mg capsule of 3-amino-1-propanesulfonic acid, form A is described below.

Capsules of 150 mgs of 3-amino-1-propanesulfonic acid, form A, are manufactured using the formulation shown in Table 7. The coating is applied through several process steps using evaporation of purified water.

TABLE 7

| | | | Quantity | |
|---|---|---|---|---|
| | | | per tablet | Proportion |
| Ingredient | Grade | Function | (mg) | (%) |
| Core: | | | | |
| 3-amino-1-propanesulfonic acid, form A | MS* | Active ingredient | 150.00 | 28.6 |
| Silicated mycrocrystalline cellulose | NF | Glidant/Diluent | 210.53 | 40.1 |
| Dibasic calcium phosphate | USP | Filler | 95.69 | 18.2 |
| Hydroxypropylmethylcellulose (HPMC) | USP | Drug Release Modifier | 52.50 | 10.0 |
| Starch ® 1500 | NF | Binder/Desintegrant | 8.33 | 1.6 |
| Stearic acid powder | NF | Lubricant | 5.25 | 1.0 |
| Magnesium stearate | NF | Lubricant | 2.70 | 0.5 |
| Weight: | | | 525.00 | 100.0 |
| Coating**: | | — | | |
| Opadry ® II White | MS* | Subcoat | 10.50 | 2.0 |
| Acryleze ® | MS* | Enteric Coat | 52.50 | 10.0 |
| Opadry ® II White | MS* | Topcoat | 10.50 | 2.0 |
| Total Weight: | | | 598.50 | 114.0 |

*MS: Manufacturer's Standard, NF: National Formulary; USP: United States Pharmacopoeia.

### Example 15

### Unit Formula of 150 mg Modified-Release Coated Tablets Containing 3-Amino-1-Propanesulfonic Acid, Form B

A pharmaceutical composition is formulated as described in Example 14 with 3-amino-1-propanesulfonic acid, form B, as the active ingredient.

### Example 16

### Unit Formula of 50 mg Modified-Release Coated Tablets Containing 3-Amino-1-Propanesulfonic Acid, Form A

An example of a formulation of a 50 mg capsule of 3-amino-1-propanesulfonic acid, form A is described below.

Capsules of 50 mgs of 3-amino-1-propanesulfonic acid, form A, are manufactured using the formulation shown in Table 8. The coating is applied through several process steps using evaporation of purified water.

TABLE 8

| | | | Quantity per | Quantity per |
|---|---|---|---|---|
| | | | tablet | batch |
| Ingredient | Grade | Function | (mg) | (kg) |
| Core: | | | | |
| 3-amino-1-propanesulfonic acid, form A | MS* | Active ingredient | 50.00 | 0.500 |
| Silicated mycrocrystalline cellulose | NF | Glidant/Diluent | 174.73 | 1.746 |
| Dibasic calcium phosphate | USP | Filler | 79.42 | 0.794 |
| Hydroxypropylmethylcellulose (HPMC) | USP | Drug Release | 35.00 | 0.350 |
| | | Modifier | | |
| Starch ® 1500 | NF | Binder/Desintegrant | 5.55 | 0.056 |
| Stearic acid powder | NF | Lubricant | 3.50 | 0.036 |
| Magnesium stearate | NF | Lubricant | 1.80 | 0.018 |
| Weight: | | | 350.00 | 3.500 |
| Coating: | | — | | |
| Opadry ® II White | MS* | Subcoat | 7.00 | 0.072 |
| Acryleze ® | MS* | Enteric Coat | 35.00 | 0.360 |
| Opadry ® II White | MS* | Topcoat | 3.50 | 0.036 |
| Total Weight: | | | 395.50 | 3.974 |

*MS: Manufacturer's Standard, NF: National Formulary; USP: United States Pharmacopoeia.

### Example 17

### Unit Formula of 50 mg Modified-Release Coated Tablets Containing 3-Amino-1-Propanesulfonic Acid, Form B

A pharmaceutical composition is formulated as described in Example 16 with 3-amino-1-propanesulfonic acid, form B, as the active ingredient.

1. Crystalline 3-amino-1-propanesulfonic acid in polymorphic Form A.

2. Crystalline 3-amino-1-propanesulfonic acid in a mixture of Form A and Form B.

3. Substantially pure crystalline 3-amino-1-propanesulfonic acid in polymorphic Form A.

4. The substantially pure crystalline 3-amino-1-propanesulfonic acid of claim 3, wherein said substantially pure crystalline 3-amino-1-propanesulfonic acid is comprised of 90% or greater polymorph Form A.

5. The substantially pure crystalline 3-amino-1-propanesulfonic acid of claim 4, wherein said substantially pure crystalline 3-amino-1-propanesulfonic acid is comprised of 95% or greater polymorph Form A.

6. The substantially pure crystalline 3-amino-1-propanesulfonic acid of claim 5, wherein said substantially pure crystalline 3-amino-1-propanesulfonic acid is comprised of 97% or greater polymorph Form A.

7. Substantially pure crystalline 3-amino-1-propanesulfonic acid in polymorphic Form B.

8. The substantially pure crystalline 3-amino-1-propanesulfonic acid of claim 7, wherein said substantially pure crystalline 3-amino-1-propanesulfonic acid is comprised of 90% or greater polymorph Form B.

9. The substantially pure crystalline 3-amino-1-propanesulfonic acid of claim 8, wherein said substantially pure crystalline 3-amino-1-propanesulfonic acid is comprised of 95% or greater polymorph Form B.

10. The substantially pure crystalline 3-amino-1-propanesulfonic acid of claim 9, wherein said substantially pure crystalline 3-amino-1-propanesulfonic acid is comprised of 97% or greater polymorph Form B.

11. The crystalline 3-amino-1-propanesulfonic acid of claim 1, wherein said crystalline 3-amino-1-propanesulfonic acid is characterized by XRPD peaks at one or more of the following °2θ values: 17.1, 21.3, and 24.7.

12. The crystalline 3-amino-1-propanesulfonic acid of claim 1, wherein said crystalline 3-amino-1-propanesulfonic acid is characterized by FT-IR peaks at one or more of the following wavelengths: 789 cm⁻¹ and 833 cm⁻¹.

13. The crystalline 3-amino-1-propanesulfonic acid of claim 1, wherein said crystalline 3-amino-1-propanesulfonic acid is characterized by an FT-Raman peak at 790 cm⁻¹.

14. The crystalline 3-amino-1-propanesulfonic acid of claim 7, wherein said crystalline 3-amino-1-propanesulfonic acid is characterized by XRPD peaks at one or more of the following °2θ values: 17.3 and 25.3.

15. The crystalline 3-amino-1-propanesulfonic acid of claim 7, wherein said crystalline 3-amino-1-propanesulfonic acid is characterized by FT-IR peaks at one or more of the following wavelengths: 803 cm⁻¹ and 843 cm⁻¹.

16. The crystalline 3-amino-1-propanesulfonic acid of claims 7, wherein said crystalline 3-amino-1-propanesulfonic acid is characterized by an FT-Raman peak at 802 cm⁻¹.

17. A pharmaceutical composition comprising crystalline 3-amino-1-propanesulfonic acid of claim 1.

18. The pharmaceutical composition of claim 17, wherein said composition further comprises a pharmaceutically acceptable carrier.

19. The pharmaceutical composition of claim 18, wherein said composition comprises the ingredients described in Example 12, Example 13, Example 14, Example 15, Example 16, Example 17 or Example 18.

20-24. (canceled)

25. The pharmaceutical composition of claim 17, wherein said pharmaceutical composition comprises an effective amount of crystalline 3-amino-1-propanesulfonic acid to treat an Aβ-amyloid related disease.

26. The pharmaceutical composition of claim 25, wherein said Aβ-amyloid related disease is Alzheimer's disease or CAA.

27. (canceled)

28. A method for treating an Aβ-amyloid related disease in a subject, comprising administering to said subject, in need thereof, an effective amount of a crystalline 3-amino-1-propanesulfonic acid of claim 1, such that said Aβ-amyloid related disease is treated in said subject.

29. The method of claim 28, wherein said subject is a human.

30. The method of claim 28, wherein said Aβ-amyloid related disease is Alzheimer's disease or CAA.

31. (canceled)
